(19) United States
(12) Patent Application Publication
(10) Pub. No.: US 2013/0337090 A1
(43) Pub. Date: Dec. 19, 2013

(54) PLANT EXTRACTS FOR TREATING BURNS AND CHRONIC WOUNDS

(71) Applicant: Shirli Doron, Givat Ze'ev (IL)

(72) Inventor: Shirli Doron, Givat Ze'ev (IL)

(73) Assignee: SKIN MATRIX LTD., Rishon Lezion (IL)

(21) Appl. No.: 13/883,976

(22) PCT Filed: Nov. 7, 2011
(86) PCT No.: PCT/IL11/00865

§ 371 Date: May 7, 2013

Related U.S. Application Data
(60) Provisional application No. 61/410,901, filed on Nov. 7, 2010.

(51) Int. Cl. A61K 36/185; A61K 36/71; A61K 45/06; A61K 36/30; A61K 35/74; A61K 36/73; A61K 36/232; A61K 36/481

(52) U.S. Cl. CPC A61K 36/185; A61K 36/232; A61K 36/71; A61K 36/481; A61K 36/30; A61K 35/74; A61K 36/73; A61K 45/06
USPC 424/726; 424/773; 424/757; 424/765

(57) ABSTRACT

The present invention provides compositions and methods for treating burns and chronic wounds, particularly compositions comprising an oil extract or oily suspension of a combination of plant material derived from several plant species for treating burns and chronic sores.

### FIELD OF THE INVENTION

The present invention provides compositions and methods for treating burns and chronic wounds, particularly compositions comprising a combination of plant extracts for treating burns and chronic sores including pressure sores.

### BACKGROUND OF THE INVENTION

In the United States alone almost 8 million people per year have burns or suffer from chronic, non-healing wounds (Singer & Clark (1999) N. Engl. J. Med. 341(10):738-746). Individuals with underlying disease tend to suffer from chronic wounds, and impaired wound healing is a hallmark of a variety of medical conditions.

Chronic wounds generally fall into three principal categories: venous stasis ulcers, diabetic ulcers and pressure ulcers. While the prevalence of these conditions varies, it is estimated that as many as 12 million people in the developed countries are afflicted with all forms of chronic wounds.

Open cutaneous wounds normally heal by a process that comprises six major components: (i) inflammation; (ii) fibroblast proliferation; (iii) blood vessel proliferation; (iv) connective tissue synthesis; (v) epithelialization; and (vi) wound contraction. The rate of chronic and acute wound healing can be delayed or impaired by a number of factors (exogenous and endogenous) and a variety of medical conditions. Examples include infection, ulceration—particularly through diabetes or continuous pressure, circulation problems associated with vascular disease, malnutrition, stress, cancer radiotherapy and/or chemotherapy, compromise of the immune system or due to the normal aging process. At present there is a clear need for therapeutic and cosmetic approaches that promote wound healing processes.

Heating of the skin, however brief, can cause damage to the cells of its living tissue. Such damage typically is referred to as a burn. Generally, skin burns are categorized into degrees that indicate the depth of the burn injury. First degree burns cause redness of the skin and affect only the epidermis. Such burns heal quickly, but the damaged skin may peel away after a day or two. “Sunburn” is an example of a first degree burn. A second degree burn damages the skin more deeply, extending into the dermis and usually causes blistering. However, some of the dermis is left to recover. A third degree burn destroys the full thickness of the skin. In fourth degree burn, the burn extends beyond the skin to the fat, muscle and bone. In third and fourth degree burn, skin transplant is typically required.

The study of plants known in traditional medicines for the treatment of diverse diseases has expanded significantly during the last decades. The active principles from many medicinal plants have been extracted, the curative agents identified and their mechanisms of action determined. Plant based medicines are typically well tolerated, with less severe and a smaller range of side effects.

There is an ongoing attempt for developing plant-based compositions for treating skin diseases and chronic wounds. For example, U.S. Patent Application Publication No. 2005/0214391 discloses a topical composition for the treatment of a dermatologic disease comprising a carrier, at least one essential oil and a dermatologic active ingredient having a therapeutic effect for the dermatologic disease, wherein the essential oil and the dermatologic active ingredient have a synergistic effect in treating the disease. Oil of Angelica is disclosed as an example of essential oil.

U.S. Patent Application Publication No. 2007/0048234 discloses anti-acne kits that are useful for treating acne, especially severe cases of acne. The anti-acne kits include a vasoconstrictor and an anti-acne agent, and optionally one or more of a skin lightening therapeutic, a sealing layer, a skin cleanser, an astringent, a skin penetration enhancer, a sunscreen, and nutritional supplements that promote healing of acne lesions. Examples of anti-acne agents include extracts of Golden Seal (Hydrastis Canadensis) and of Angelica root. The Golden Seal extract may be also used as an astringent.

U.S. Patent Application Publication No. 2007/0122492 discloses plant extracts and dermatological formulations comprising one or more plant extracts that are capable of inhibiting one or more extracellular proteases. Also disclosed is the use of the plant extracts as dermatological agents suitable for the treatment or prevention of various dermatological conditions, including wrinkling or sagging of the skin, irradiation induced skin and/or hair damage, deepening of skin lines, elastotic changes in the skin, as well as for the routine care of the skin, hair and/or nails.

U.S. Patent Application publication No. 2008/0206155 discloses stable non-alcoholic foamable carriers and pharmaceutical emulsion compositions comprising an unctuous emollient, a multi-active agent, water, and a propellant with and without the addition of an active agent. The application further discloses that the active agent may be an extract or tincture of one or more beneficial agents that have beneficial properties, for example, when applied to the skin, a body surface, a body cavity or a mucosal surface. Herbal extracts may be from herbs including Angelica, Comfrey, Dandelion, Agrimony, Elecampane, Golden Seal and Nettle and wherein the extract may contain, for example, an aqueous, polar, hydrophobic or potent solvent.

U.S. Application Publication No. 2009/0004301 discloses formulations comprising plant extracts and niacin, that when combined yield an effective multi-faceted pharmaceutical approach to treating dry skin disorders, and methods of use thereof to prevent and/or treat dyshidrosis (pompholyx) and related skin diseases including contact dermatitis, eczema, palmoplantar pustulosis and skin infections incurred by invasive pathogens such as mold, fungus and bacteria. The active ingredients within the formula include a combination of dry, aqueous, acid and alcohol extracts of Black Walnut Hull (Juglans nigra), Wormwood (Artemisia absinthium), Tumeric Rhizome (Curcuma longa), Garlic (Allium sativum), two or more herbal antibacterial agents from the group consisting of Chamomile (Matricaria Chamomile), Licorice Root (Glycyrrhiza glabra), St Johns Wort (Hypericum perforatum), Clove (Syzygium aromaticum), Nutmeg (Myristica fragans), Ginger (Zingiber officinale), Frankincense (Boswellia carteri) and Myrrh (Commiphora molmol), further combined with Aloe Vera and niacin.

U.S. Pat. No. 7,682,617 discloses phytoceutical compositions for the prevention and treatment of inter alia, dermal disorders. A specific combination of extracts of plants is taught; for treating skin disorders, the composition comprises effective amounts of Rhaponticum carthamoides, Rhodiola rosea, Szchisandra chinensis, Angelica sinensis, Astragalus membranaceus, Echinacea angustifolia, Echinacea angustifolia radix, Echinacea purpurea, Echinacea purpurea radix, Ganoderma lucidum mushroom, Hydrastis canadensis, Lepidium meyenii, Panax ginseng, Silibum marianum, Shark Cartilage, Tabebuia avellanedae, Uncaria Tomentosa, Equisetum arvense, Fulvic Acid-Shilajit, Hydrocotile asiatica, Picrorhiza kurroa, Smilax officinialis and Vaccinium mirtyllus, together with pharmaceutically acceptable excipients. The disclosed combination has synergistic effects, with minimal side effects.

However, despite the beneficial medicinal qualities of many plants, they are individually insufficient to overcome chronic sores. While synthetic drugs can be highly effective, their use is often hampered by severe side effects. Thus, there is a great need for, and it would be highly advantageous to have compositions of plant extracts providing sufficient efficacy together with high patient tolerance.

### SUMMARY OF THE INVENTION

The present invention discloses entirely natural compositions comprising oil extracts or suspension of roots, optionally further comprising oil extract or suspension of leaves of several plant species, effective in treating burns and chronic sores. The compositions of the present invention are highly effective in promoting healing of sever (stages three and four) burns and wounds while not having deleterious side effects.

The present invention is based in part on the unexpected discovery that applying a composition comprising an oil extract of a mixture of ground roots of Harpagophytum procumbens (Devil's claw), Hydrastis canadensis (Golden Seal) and Angelica archangelica (Angelica) to the area of acute pressure ulcer resulted in significant alleviation of the ulcer symptoms and in wound repair. It has been also observed that applying the composition to burns, including third and four degree burns resulted in skin healing. The compositions of the present invention have been also shown to be effective in treating and alleviating pathological fissure and hemorrhage conditions.

Without wishing to be bound by any particular theory or mechanism of action the ability of the composition of the present invention to promote wound and burn healing may be attributed to the combination of its properties as disinfectant and promoter of epithelialization.

Although several of the plant species used according to the teachings of the present invention have been previously reported as sources for skin formulations, the particular combination disclosed herein shows a synergistic and a long term effect in alleviating burn symptoms and healing chronic sores.

The compositions of the present invention are advantageous over hitherto known composition for treating wounds, either herbal preparations or conventional drugs, in that minimal number of applications is required, such that the pain and discomfort associated with treating the wound is reduced to minimum. Without wishing to be bound by any specific theory or mechanism of action, the minimal number of administration required is due to the dual activity of the composition, which is highly effective in disinfecting the wound and its area and in healing the damaged tissue.

Thus, according to one aspect, the present invention provides a composition comprising as an active ingredient an oil extract or oily suspension of a combination of ground roots of Harpagophytum procumbens (Devil's claw) and Angelica archangelica (Angelica). According to some embodiments, the active ingredient further comprises an oil extract or oily suspension of ground roots of Hydrastis canadensis (Golden Seal).

According to certain embodiments, the composition further comprises oil extract or oily suspension of at least one of ground root of Astragalus membranaceous (Astragalus); ground leaves of Symphytum officinale (Comfrey); ground leaves of Urtica dioica (Stinging Nettle) or any combination thereof. According to additional embodiments, the composition further comprises an oil extract or oily suspension of ground leaves of Agrimonia Eupatoria (Agrimony) and ground blue-green algae.

According to certain typical embodiments, the composition of the present invention comprises as an active ingredient an oil extract or oily suspension of a combination of ground plant material comprising roots of Harpagophytum procumbens (Devil's claw), Angelica Archangelica (Angelica) and Astragalus membranaceous (Astragalus); leaves of Symphytum officinale (Comfrey) and of Urtica dioica (Stinging Nettle) and Agrimonia Eupatoria (Agrimony); and blue-green algae. According to additional typical embodiments, the active ingredient further comprises an oil extract or oily suspension of ground roots of Hydrastis canadensis (Golden Seal).

According to certain embodiments, the composition of the present invention consists of a combination of ground plant material consisting of roots of Harpagophytum procumbens (Devil's claw) and Angelica archangelica (Angelica). According to other embodiments, the composition consists of an oil extract or oily suspension of a combination of ground plant material consisting of roots of Harpagophytum procumbens (Devil's claw), Hydrastis canadensis (Golden Seal) and Angelica archangelica (Angelica).

According to additional embodiments, the composition of the present invention consists of an oil extract or oily suspension of a combination of ground plant material consisting of roots of Harpagophytum procumbens (Devil's claw) Angelica Archangelica (Angelica) and Astragalus membranaceous (Astragalus); leaves of Symphytum officinale (Comfrey) and of Urtica dioica (Stinging Nettle) and Agrimonia Eupatoria (Agrimony); and ground blue-green algae. According to some embodiments, the active ingredient further consists of an oil extract or oily suspension of roots of Hydrastis Canadensis (Golden Seal).

According to some embodiments, the composition of the present invention consists of an oil extract or oily suspension of a combination of ground plant material consisting of roots of Harpagophytum procumbens (Devil's claw), Hydrastis Canadensis (Golden Seal), Angelica Archangelica (Angelica) and Astragalus membranaceous (Astragalus); leaves of Symphytum officinale (Comfrey) and of Urtica dioica (Stinging Nettle) and Agrimonia Eupatoria (Agrimony); and ground blue-green algae.

According to certain embodiments, the oil is selected from the group consisting of a mineral oil, a vegetable oil and combinations thereof. In particular embodiments, the vegetable oil is selected from the group consisting of wheat germ oil, almond oil, canola oil, coconut oil, corn oil, cottonseed oil, grape seed oil, olive oil peanut oil, saffron oil, sesame oil, soybean oil, and combinations thereof.

According to certain currently typical embodiments, the oil is wheat germ oil. According to additional embodiments, the plant material used according to the teachings of the present invention is dry.

According to certain embodiments, the combination of ground roots comprises 10%-15% by volume with regard to the total root volume (v/v) of Devil's claw, 5%-8% by volume roots of Golden Seal and 70%-85% by volume roots of Angelica with regard to the total volume of the roots.

According to currently typical embodiments, the composition comprises 2%-3% by volume with regard to the total volume of the plant material (v/v) roots of Devil's claw; 0.5%-1.5% roots of Golden Seal; 15%-17% roots of Angelica; 15%-17% roots of Astragalus; 15%-17% leaves of Comfrey; 7%-9% leaves of Stinging Nettle; 15%-17% leaves of Agrimony and 20%-25% blue-green alga, all percentages by volume with regard to the volume of the plant material (v/v).

According to certain embodiments, all the dry plant material is ground to a fine powder before it is mixed with oil. According to other embodiments, the dry plant material is ground within oil to form a fine suspension.

According to further embodiments, the composition further comprises at least one additional active ingredient selected from the group consisting of an anti-inflammatory agent, analgesic agent, anesthetic agent, antihistamine, disinfectant, antibiotic agent and a drug effective in wound healing.

According to further embodiments, the composition further comprises additives useful in the dermatological or pharmaceutical fields, including, but not limited to, fats, emulsifiers and co-emulsifiers, hydrophilic or lipophilic gelling agents, preservatives, solvents, fragrances, fillers, hydrophilic and lipophilic filters, neutralizers, penetration-enhancing agents and polymers. The quantities of these various additives are those conventionally used in dermatological preparations as is known to a person skilled in the art.

Being an oil extract or oily suspension of all natural plant material the composition of the present invention may be directly applied to the wounded skin. Additionally or alternatively, the composition can be further formulated for topical application as is known to a person skilled in the art as long the formulation preserves the extract activity. According to certain embodiments, the composition is formulated to a form selected from the group consisting of a cream, an ointment, a lotion, a gel, an emulsion including water in oil or oil in water emulsion, multiple emulsion, silicone emulsion, microemulsion or nanoemulsion. According to further embodiments, the formulated composition is embedded in a wound dressing.

According to certain embodiments, the composition comprises a pharmaceutically or dermatologically acceptable carrier suitable for forming a gel preparation, including but not limited to methyl cellulose, agarose, dextrans, polysaccharides, gelatine, aloe vera extract Acemannan-beta-(1,4)-linked acetylated mannan or other pharmaceutically acceptable vehicles.

The present invention now shows that applying the compositions of the present invention to a wound or a burn results in an immediate initiation of the wound healing process leading to significant improvement in the burn or wound stage until a complete cure.

Thus, according to additional aspect, the present invention provides a method for treating a burn or chronic wound comprising topically administering to a subject in need thereof an effective amount of a composition comprising an oil extract or oily suspension of a combination of ground roots of Harpagophytum procumbens (Devil's claw) and Angelica Archangelica (Angelica), thereby promoting the healing of the burn or the wound.

According to some embodiments, the composition further comprises an oil extract or oily suspension of ground roots of Hydrastis canadensis (Golden Seal).

According to certain embodiments, the composition further comprises oil extract or oily suspension of at least one of root of Astragalus membranaceous (Astragalus); leaves of Symphytum officinale (Comfrey); leaves of Urtica dioica (Stinging Nettle) or any combination thereof. According to additional embodiments, the composition further comprises oil extract or oily suspension of ground leaves of Agrimonia Eupatoria (Agrimony) and ground blue-green algae.

According to certain typical embodiments, the composition of the present invention comprises an oil extract or oily suspension of a combination of ground roots of Harpagophytum procumbens (Devil's claw), Angelica Archangelica (Angelica) and Astragalus membranaceous (Astragalus); ground leaves of Symphytum officinale (Comfrey), Urtica dioica (Stinging Nettle) and Agrimonia Eupatoria (Agrimony); and ground blue-green algae. According to certain embodiments, the oil is wheat germ oil. According to additional typical embodiments, the composition further comprises an oil extract or oily suspension of ground roots of Hydrastis canadensis (Golden Seal).

According to certain embodiments, administering the composition comprises covering the wound or burn area with said composition, and closing the treated skin area with a wound dressing. According to other embodiments, administering the composition comprises injecting the composition into the wounded tissues and closing the treated skin area with a wound dressing.

Any wound dressing as is known in the art can be used according to the teachings of the present invention. According to certain embodiments, the wound dressing comprises the composition of the present invention. According to these embodiments, the wound dressing is applied directly to the burn or wound area.

According to certain embodiments, the wound dressing is left on the treated skin for at least 3 days and then removed. According to these embodiments, after removing the wound dressing, the composition is administered again at least once. According to certain embodiments, the composition is further administered up to the complete healing of the burn or wound, with or without covering the wound with a wound dressing. The number of repeated administrations would depend on the severity of the initial burn or wound. Typically, one to 15 repeated administrations at a regime of once in about 4-7 days is required for full recovery of sever (stage 4) burn or wound. A significant advantage of the method of the present invention is the need to administer the composition once in 4-7 days, compared to several administrations per day for commonly used wound-treating compositions, thus reducing significantly the pain and discomfort associated with treating wounds.

A wide variety of wounds can be treated with the composition of the invention including, but not limited to, full and partial thickness burn wounds, sunburns, frostbite; ulcerative lesions such as pressure (decubitus) ulcers and varicose, stasis and trophic ulcers; wounds associated with surgical procedures such as amputation, incision, circumcision and episiotomy; traumatic and pyogenic wounds; vaginitis; cervicitis; pilonidal cyst wounds; pathological fissure and pathological hemorrhoids.

According to certain embodiments, the wounds are associated with states in which the normal wound repair ability is weakened. According to typical embodiments, the wounds are associated with states such as diabetes and/or occur in patients in their older age or during steroid treatment or chemotherapy. According to typical embodiments, the wound is selected from the group consisting of diabetic foot ulcers, neuropathic ulcers including neuropathic forefoot ulcers, diabetic pressure ulcers or diabetic venous ulcers; bed sores or pressure ulcers associated with long-term disability, each representing a separate embodiment of the present invention.

The composition amount and administering regimen will be set according to the burn or wound type and severity and parameters of the subject including age, gender and general health as measured by a skilled practitioner.

According to certain embodiments, the chronic wound is associated with a state in which the normal wound repair ability is weakened. According to additional embodiments, the chronic wound is associated with diabetes and/or occurs in patients in their older age and/or during steroid treatment.

According to currently typical embodiments, the chronic wound is a pressure ulcer. According to one embodiment, the pressure ulcer is associated with long-term disability.

Other objects, features and advantages of the present invention will become clear from the following description and drawings.

### BRIEF DESCRIPTION OF THE DRAWINGS

FIG. 1 shows pressure ulcer in the leg before (FIG. 1A) and after (FIG. 1B) treatment with the composition of the invention.

FIG. 2 shows pressure ulcer in the sacrum before (FIG. 2A) and after (FIG. 2B) treatment with the composition of the invention.

FIG. 3 shows diabetic ulcer before (FIG. 3A) and after (FIG. 3B) treatment with the composition of the invention.

### DETAILED DESCRIPTION OF THE INVENTION

### Definitions

The term “extract” when used in connection with a plant, refers to one or more active agents, or a composition containing such, that is obtained from the plant, or a portion thereof. According to typical embodiments, the extracts of the present invention are obtained from the plant roots or leaves. The extracts of the invention are typically referred to as crude extracts, as no refining or selection of particular fractions or ingredients is performed. The extracts of the present invention are typically first extracts, i.e. the extraction oil is added to the plant material only once and the plant material is removed from the resulting solution or suspension after a certain incubation time. According to typical embodiments of the invention, extraction is performed with wheat germ oil. The term “oily suspension” as used herein refers to the extract as described above, from which the plant material is not removed.

The term “dry material” refers to roots or leaves from which water is evaporated until no further change in the root or leaf weight is observed. Any method as is known to a person skilled in the art for drying plant material may be used according to the teachings of the present invention. The term “dry weight” as used herein refers to the weight of the dry material.

The term “root” is used herein in its broadest scope, including any non-aerial part of the plant. It is to be explicitly understood that the term “root” as used herein encompasses bulbs, corms, tubers, rhizome roots etc.

As used herein, the term “burn” refers to an injury to tissues caused by heat, cold, chemicals, electricity, or irradiation effects.

The term “chronic wounds” as used herein refers to clinical conditions having characteristic symptoms wherein the wounds do not heal for a prolonged time and frequently have a strong tendency to recurrence. The chronic wounds usually are associated with states in which the normal wound healing ability is weakened, such as diabetes, patients in their older age, patients with limited ability of movement, or patients during steroid treatment or chemotherapy. Thus, patients with such badly healing wounds are typically those suffering from diabetic ulcers, including diabetic foot ulcers, diabetic neuropathic ulcers, including neuropathic forefoot ulcers, diabetic pressure ulcers or diabetic venous ulcers, as well as patients with limited movement ability suffering from bed sores. Bed sores usually affect peoples who stay in one place for prolonged time for any reason.

As used herein, the terms “severity of the burn” or “severity of the wound” refer to one or more parameters used to classify the burn or wound, such as the degree of burn/wound, the percentage of the total body surface area affected by the burn/wound and the depth of the burn/wound.

The severity of a wound or a burn is typically classified into stages and degrees, respectively.

Stage one wounds do not have any visible skins cuts. However, the skin covering the wound can be remarkably different from the surrounding area. The differences may be changes in temperature, firmness, or color of the skin. The wound may also be painful or itchy.

In a Stage two wound the top most layers of skin is severed (epidermis and dermis). There may be some drainage.

Stage three wounds are deeper than stage 2 wounds. They typically go down to the “fat” layer (subcutaneous), but do not extend any further. There may be dead tissue and drainage.

Stage four wounds are very serious. These wounds are characterized by going as far down as the bone and muscle. Dead tissue and drainage are almost always present. Often, at this stage, the wound is also infected.

First degree burns result in some redness and swelling of the injured part, without necrosis of any tissue or the formation of blisters.

Second degree burns show a variable destruction of parts of the dermis so that blistering occurs. Healing by regeneration in such superficial burns does not necessitate skin grafting, unless secondary infections ensue.

Third degree burns are marked by complete destruction of a skin region, including the necrosis of accessory skin structures such as hair and sweat glands. A brownish-black eschar (a dry scab or slough) marks the destroyed tissue. In deep third degree burns, also classified as fourth degree burns, tissue is destroyed to the level of or below the deep fascia lying beneath the subcutaneous fat and connective tissue of the body. Muscle, bone, deeper nerves, and even organs may be injured or destroyed by this severe degree of burn.

The terms “synergistic” or “synergy” is used herein to mean that the effect is more than its additive property.

Concentrations, amounts, ratios, and other numerical data may be expressed or presented herein in a single number or a range format. It is to be understood that the range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

### Preferred Modes for Carrying Out the Invention

The present invention discloses entirely natural compositions comprising oil extracts or oily suspension of roots optionally further comprising oil extract or oily suspension of leaves of several plant species, effective in treating burns and chronic sores including pressure sores. The compositions of the present invention are highly effective in promoting burn and wound healing while not having deleterious side effects and being comfort to the patient.

According to one aspect, the present invention provides a composition comprising an oil extract or oily suspension of a combination of ground roots of Harpagophytum procumbens (Devil's claw) and Angelica archangelica (Angelica). According to some embodiments, the composition further comprises an oil extract or oily suspension of ground roots of Hydrastis canadensis (Golden Seal).

According to certain embodiments, the composition further comprises oil extract or oily suspension of at least one of ground root of Astragalus membranaceous (Astragalus); ground leaves of Symphytum officinale (Comfrey); ground leaves of Urtica dioica (Stinging Nettle) or any combination thereof. According to additional embodiments, the composition further comprises an oil extract or oily suspension of ground leaves of Agrimonia Eupatoria (Agrimony) and ground blue-green algae.

According to certain typical embodiments, the composition of the present invention comprises as an active ingredient an oil extract or oily suspension of a combination of ground plant material comprising roots of Harpagophytum procumbens (Devil's claw), Angelica Archangelica (Angelica) and Astragalus membranaceous (Astragalus); leaves of Symphytum officinale (Comfrey), Urtica dioica (Stinging Nettle) and Agrimonia Eupatoria (Agrimony); and blue-green algae. According to additional typical embodiments, the active ingredient further comprises an oil extract or oily suspension of roots of Hydrastis canadensis (Golden Seal).

According to additional embodiments, the composition of the present invention consists of an oil extract or oily suspension of a combination of ground plant material consisting of ground roots of Harpagophytum procumbens (Devil's claw); ground roots of Angelica Archangelica (Angelica); ground roots of Astragalus membranaceous (Astragalus); ground leaves of Symphytum officinale (Comfrey); ground leaves of Urtica dioica (Stinging Nettle); ground leaves Agrimonia Eupatoria (Agrimony); and ground blue-green algae.

According to some embodiments, the composition of the present invention consists of an oil extract or oily suspension of a combination of ground plant material consisting of roots of Harpagophytum procumbens (Devil's claw), Hydrastis Canadensis (Golden Seal), Angelica Archangelica (Angelica) and Astragalus membranaceous (Astragalus); leaves of Symphytum officinale (Comfrey) and of Urtica dioica (Stinging Nettle) and Agrimonia Eupatoria (Agrimony); and ground blue-green algae.

Plant Characteristics

Harpagophytum procumbens (Devil's claw): The major known chemical constituents of Harpagophytum are iridoid glycosides (primarily harpagoside, harpagide, and procumbide), sugars (mainly the tetrasaccharide stachyose), triterpenoids (oleanolic and ursolic acid), phytosterols (primarily beta-sitosterol), aromatic acids (caffeic, cinnamic, and chlorogenic acids), and flavonoids such as luteolin and kaempferol. Harpagoside, harpagide, and procumbide, found in the tubers of the plant, appear to be the most therapeutically important constituents. H. procumbens has been used as an analgesic, a remedy for fevers and allergies, as a diuretic and sedative, for treating dyspepsia, for appetite stimulation, and for treating degenerative disorders of the musculoskeletal system. It has also been used for liver and kidney disorders, as an oxytocic, and as a topical agent to treat wounds and skin rashes.

Hydrastis canadensis (Golden seal, yellow root, turmeric root) contains mainly isoquinoline alkaloids (xanthopuccine, berberine, hidrastine, hidrastanine, beta-hydrastine, canadine and canadaline). These confer anti-inflammatory, bacteriostatic, bacteriocidal, and vasodilator effects. In general, its antibacterial action is directed to metabolic inhibition, inhibition of the formation of enterotoxins, and inhibition of bacterial adhesion. It causes vasodilatation by inhibiting smooth muscle contraction and inhibiting platelet aggregation. At least 34 active ingredients from this plant has been proposed for therapeutic use.

Angelica archangelica (Dong quai or Angelica, also Angelica sinensis, Angelica pubescens and Angelica sylvestris) is known to contain terpenes (mainly β-phellandrene, with β-bisabolene, .β-caryophyllene, β-phellandrene, α- and β-pinene, limonene, linalool, borneol, acetaldehyde, menthadienes and nitromenthadienes), macrocyclic lactones (including tridecanolide, 12-methyl tridecanolide, pentadecanolide), phthalates (such as hexamethylphthalate), coumarins (especially furocoumarin glycosides such as marmesin and apterin), angelicin and byakangelicin derivatives (osthol, umbelliferone, psoralen, bergapten, imperatoren, xanthotoxol, xanthotoxin, oxypeucedanin and more), as well as various sugars, plant acids, flavonoids, and sterols. These components have vasodilatation activity, increase coronary flow and are anti-thrombotic.

Astragalus membranaceus (Astragalus; Huang-Qi): This plant is known to contain three main types of active ingredients. Isoflavones, which act as anti-oxidants; astragalans, which act as immuno-stimulants and anti-inflammatory by stimulating the phagocytic activity of macrophages, of the cytotoxic response of T and NK lymphocytes and of the production and activity of interferon; and astragalans which act as modulators of the hypothalamus-hypophysis-adrenal axis response.

Symphytum officinale (Comfrey): The herb contains allantoin, a cell proliferation agent that accelerates the natural replacement of body cells. Comfrey was used in an attempt to treat a wide variety of ailments ranging from bronchial problems, broken bones, sprains, arthritis, gastric and varicose ulcers, severe burns, acne and other skin conditions. It was reputed to have bone and teeth building properties in children, and have value in treating “many female disorders”. Constituents of comfrey also include mucilage, steroidal saponins, tannins, pyrrolizidine alkaloids, inulin, and proteins.

Urtica dioica (Stinging Nettle): Stinging nettle has been used for hundreds of years to treat painful muscles and joints, eczema, arthritis, gout, and anemia. Today, many people use it to treat urinary problems during the early stages of an enlarged prostate (called benign prostatic hyperplasia or BPH), for urinary tract infections, for hay fever (allergic rhinitis), or in compresses or creams for treating joint pain, sprains and strains, tendonitis, and insect bites. Stinging nettle products are usually made from the leaves and stems, and sometimes the roots. Root preparations are used to relieve symptoms of BPH.

Agrimonia Eupatoria (Common Agrimony, Church steeples or Sticklewort): As of today, Agrimony is prescribe by herbalists for disorders of the kidneys, liver and bladder, and for irritable bowel syndrome. It is a mild astringent. Agrimony is also considered a very useful agent in skin eruptions and diseases of the blood, pimples, blotches, etc.

Blue-green algae (Spirulina) are particularly known for their nutritional value. The algae are rich in vitamin B12, iron and essential fatty acids. They have high protein content and are used as a protein source for vegetarian. The algae are also known as effective in stabilizing blood sugars.

According to certain embodiments, the oil is wheat germ oil. According to additional embodiments, the roots and leaves used according to the teachings of the present invention are in a dry form.

According to certain embodiments, the process for preparing the compositions of the present invention includes the following general steps:

According to the plant species, dry roots or leaves are obtained. The plant material should be completely dry (i.e. there is no change in its weight under further heating). The dried plant material is then ground manually or mechanically to obtain as fine powder as possible, that is then mixed with the oil to produce fine suspension. In case the source plant material appears to contain impurities, the powder is mildly heated. Alternatively, the dried plant material is mixed with the oil and then the size of the plant material is reduced via several steps to obtain a fine suspension. When the plant material goes through size reduction, the temperature is kept below 40° C. through all the size reduction steps.

Typically, the oil is wheat germ oil. The oily suspension is stirred and then heated to about (and typically no more than) 40° C. The suspension is then cooled, typically at room temperature.

According to certain embodiments, the first suspension is composed of roots of Harpagophytum procumbens (Devil's claw), Hydrastis canadensis (Golden Seal), Angelica archangelica (Angelica) and Astragalus membranaceous (Astragalus); and leaves of Symphytum officinale (Comfrey) and Urtica dioica (Stinging Nettle). After cooling, a suspension containing oil and powder of leaves of Agrimonia Eupatoria (Agromony) and blue green algae is added. According to other embodiments, the entire ingredients are mixed together to form single suspension.

The final suspension is kept at room temperature in hermetically closed, non-transparent container, typically made of glass.

It is to be understood that various conditions may be used in the course of the present extraction method, in order to obtain the composition of the present invention, as long as its activity is kept. Specific conditions, such as the amount of oil added and the heating temperature, as well as the time the entire composition is kept at room temperature may be varied.

According to certain embodiments, the suspension is filtered through a sieve to separate the plant material from the oil containing the desirable bioactive agents. Such filtering may employ a number of different suitable filter types, from cookware strainer to paper filters, nylon mesh, etc. The filtration process may be accomplished without any specific apparatus or may employ any filtration process and apparatus known to those of ordinary skill in the art, such as reduced pressure vacuum filtration. Once the oil is separated from the plant material, the plant material is discarded. According to currently typical embodiments, the plant material is not reclaimed for further extraction. However, it is to be explicitly understood that a process comprising further extraction cycles is encompassed within the teachings of the present invention.

The oily suspension containing the plant material or the filtered extract is now ready for application to the affected skin. Being an oil preparation, it can be directly administered to the wound or the burn. Alternatively, the extract or oily suspension can be further formulated as pharmaceutical or dermatological compositions.

Thus, according to certain embodiments, the present invention provides pharmaceutical or dermatological composition comprising the suspension or extract of the invention, further comprising a pharmaceutically or a dermatologically acceptable diluent, excipient or carrier.

As used herein a “pharmaceutical composition” or “dermatological composition” refers to the extract of the present invention with other chemical components such as physiologically suitable carriers and excipients. The purpose of a pharmaceutical/dermatological composition is to facilitate administration of the plant-derived extract of the invention to an organism.

Pharmaceutical or dermatological compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes. Proper formulation is dependent upon the route of administration chosen.

According to certain embodiments, the pharmaceutical composition is formulated for topical injection into the wound. According to other embodiments, the pharmaceutical or dermatological composition is formulated for topical application onto the wound.

According to certain embodiments, the composition is formulated to a form selected from the group consisting of a cream, an ointment, a lotion, a gel, an emulsion including water in oil or oil in water emulsion, multiple emulsion, silicone emulsion, microemulsion or nanoemulsion. Each possibility represents a separate embodiment of the invention.

According to certain embodiments, the composition is formulated in the form of a gel. In some embodiments, the compositions are incorporated into or applied onto a wound dressing. Any wound dressing as is known to a person skilled in the art can be used according to the teachings of the present invention.

Unexpectedly, the present invention now shows that administering the extract or oily suspension of the present invention to a wound, including to an infected sever stage four wound results in enhanced healing of the wound after administration of the composition once in 3-7 days for a period of about two months. This phenomenon is highly advantageous to the subject as it reduces the pain associated with repeated treatment (1-6 times a day) of the open wound with hitherto known compositions.

Thus, according to additional aspect, the present invention provides a method for treating a burn or chronic wound comprising topically administering to a subject in need thereof an effective amount of a composition comprising an oil extract or oily suspension of a combination of ground roots of Harpagophytum procumbens (Devil's claw) and Angelica Archangelica (Angelica), thereby promoting the healing of the burn or the wound. According to certain embodiments, the composition further comprises oil extract or oily suspension of ground roots of Hydrastis Canadensis (Golden Seal).

According to certain embodiments, the composition further comprises oil extract or oily suspension of at least one of root of Astragalus membranaceous (Astragalus); leaves of Symphytum officinale (Comfrey) leaves of Urtica dioica (Stinging Nettle) or any combination thereof. According to additional embodiments, the composition further comprises oil extract or oily suspension of ground leaves of Agrimonia Eupatoria (Agrimony) and ground blue-green algae.

According to certain typical embodiments, the composition of the present invention comprises an oil extract or oily suspension of a combination of ground roots of Harpagophytum procumbens (Devil's claw), Angelica Archangelica (Angelica) and Astragalus membranaceous (Astragalus); ground leaves of Symphytum officinale (Comfrey) and of Urtica dioica (Stinging Nettle) and Agrimonia Eupatoria (Agrimony); and ground blue-green algae. According to certain embodiments, the oil is wheat germ oil. According to additional typical embodiments, the composition further comprises an oil extract or oily suspension of roots of Hydrastis canadensis (Golden Seal).

The terms “effective amount” as used in connection with the compositions of the present invention refer to an amount of the composition that is sufficient to achieve an intended wound healing effect. Thus, “an effective amount” refers to a non-toxic, but sufficient amount of the composition to achieve therapeutic results in treating a burn or a wound as described herein. Various biological factors may affect the ability of a substance to perform its intended task. Therefore, an “effective amount” or a “therapeutically effective amount” may depend on such biological factors. The determination of an effective amount is well within the ordinary skill in the art of pharmaceutical, nutraceuticai, herbaceutical, and health sciences. See, for example, Meiner and Tonascia, “Clinical Trials: Design, Conduct, and Analysis,” Monographs in Epidemiology and Biostatistics, Vol. 8 (1986). According to certain aspects of the invention, the term “therapeutic effective amount” refers to the amount capable of promoting the healing of a wound or a burn, up to complete healing of the skin tissues.

The healing properties of the compositions of the present invention have been demonstrated in a number of test situations, including pressure ulcers and diabetes ulcers.

For wound healing, effectiveness is determined, among other indications, by wound contracture, increased rate of healing and/or improved healing (i.e., maintain response to tactile stimulus, less scarring, improved neovascularization, etc.)

As used herein, “subject” refers to a mammal that may benefit from the administration of the composition of the present invention; most often, the subject is a human.

The method by which the composition of the invention is administered to the wound depends, among other parameters, on the type and severity of the wound. Typically, shallow burns and wounds with large surface area are treated with topical compositions formulated for application onto the upper wound area. To stage 3-4 wounds which go deep down the skin tissues, the composition is typically administered by topical injection into the open wound. In both cases, after the first administration the wound is covered with a wound dressing and is left closed for at least three days. After the wound dressing is taken off, the healing process continuous, typically requiring application of the composition only to the margins of the wound. At times, additional application of the composition is required, with or without the coverage of the wound with a wound dressing.

The present invention further shows that the composition of the present invention is highly effective in preventing and treating infections. Without wishing to be bound by any specific theory or mechanism of action, the disinfectant properties of the extract or oily suspension of the present invention is attributed to the presence of Agrimony and blue-green alga. The present invention now discloses that direct application of a powder of a combination of ground Argrimony leaves and blue green algae is highly effective in preventing and eliminating infection.

The disinfectant properties of the extract/oily suspension of the invention, together with being non-toxic, make the extract/oily suspension of the present invention highly suitable for treating throat infection.

According to a further aspect, the present invention provides a method for treating throat infections comprising administering to a subject in need thereof a therapeutically effective amount of the composition of the present invention as described herein.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

### Oily Suspension and Extract Preparation

1. Half table spoon of Harpagophytum procumbens (Devil's claw) roots with three table spoons of Symphytum officinale (Comfrey) were ground using mortar and pestle.
2. Three tea spoons leaves of Urtica dioica (Stinging Nettle) and half table spoon roots of Hydrastis Canadensis (Golden Seal) were ground using mortar and pestle. The powder was heated for about 10 min and then cooled at room temperature.
3. Three table spoons roots of Astragalus membranaceous (Astragalus) were ground using mortar and pestle
4. Three table spoons of Angelica Archangelica (Angelica) were ground using mortar and pestle.
5. About 1.00 ml of wheat germ oil were added to the powder obtained in steps 1-4 above, and al the ingredients were well mixed. The suspension was then heated up to the point of boiling (about 5-10 min); removed from the heat and left to cool in room temperature.
6. Three table spoons leaves of Agrimonia Eupatoria (Agrimony) and eight table spoons (eight capsules) of blue green algae were ground using mortar and pestle; the resulted powder was added to about 200 ml of wheat germ oil.
7. The suspensions obtained in steps 5 and 6 were combined to produce the suspension composition of the present invention.
8. Optionally, the suspension of step 7 was filtered through a sieve to remove the plant material to produce the extract of the present invention.

### Example 2

### Promoting Wound Healing Using the Compositions of the Invention

The activity of the compositions of the present invention was evaluated by application to pressure and diabetes ulcers of several volunteers, first and second degree relatives of the inventors of the present invention.

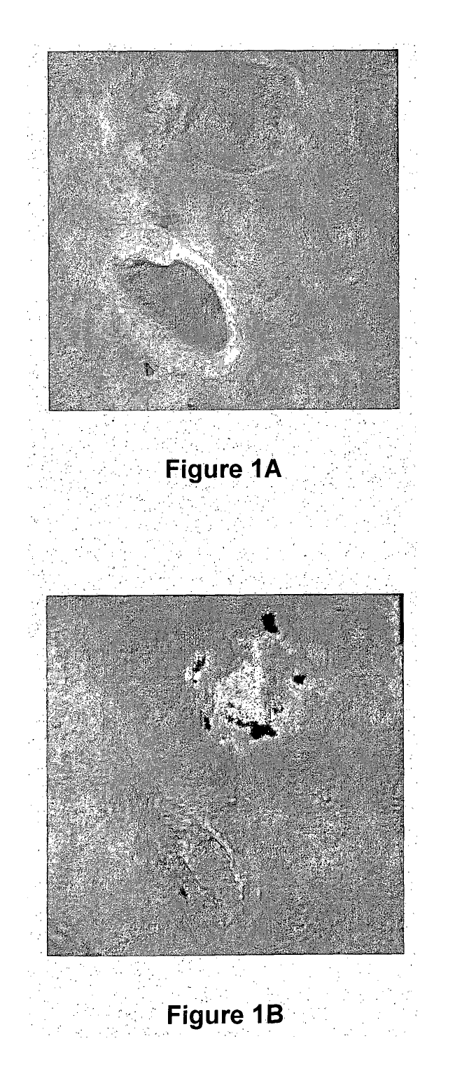

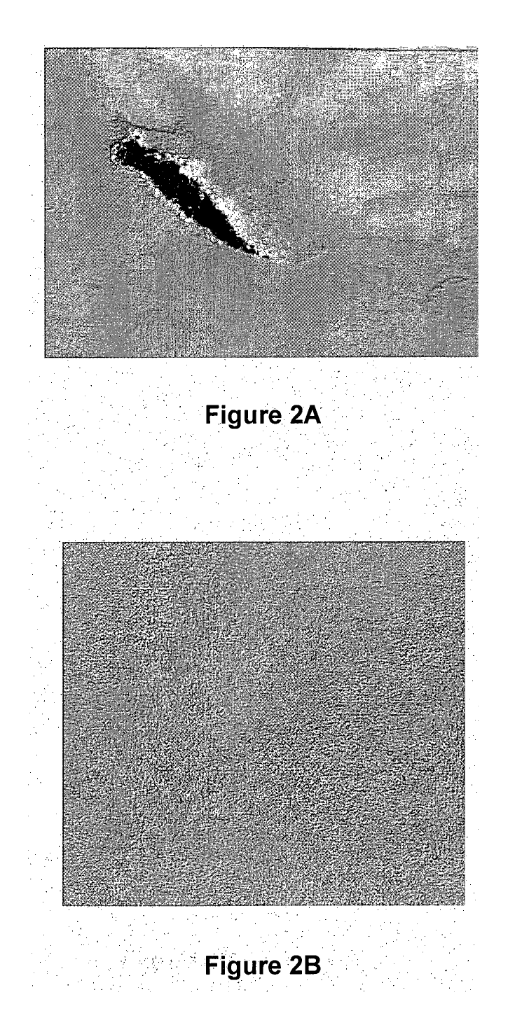

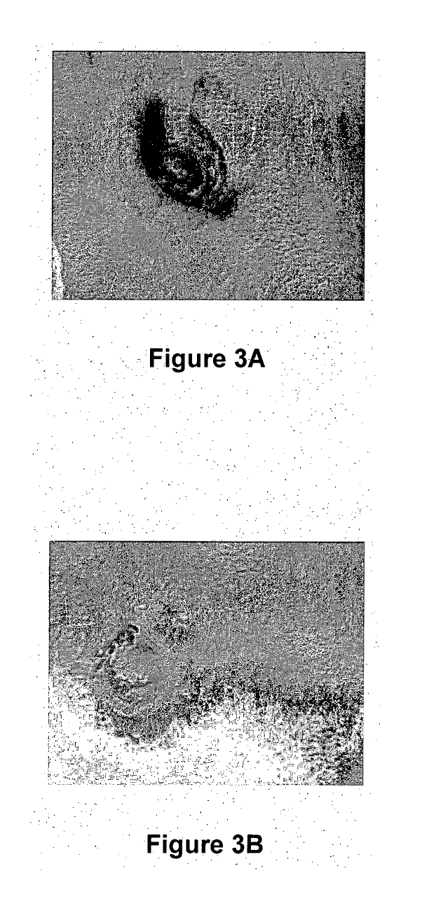

FIG. 1 shows the healing of a pressure ulcer in the leg. FIG. 2 shows the healing of a pressure wound in the sacrum. A wound at an area of about 8 cm² and depth of about 4 cm (FIG. 2A) before the treatment turned to almost invisible wound (FIG. 2B). FIG. 3 shows the healing of a diabetic ulcer.

In all cases, the wound was first disinfected with sterile cheese-cloth (gauze) pad. Then the oily suspension prepared as described in Example 1 hereinabove (including the plant material) was spread over a sterile gauze pad that was applied onto the wound, and fixed using a bandage. The wound was left covered for four days, after which the process was repeated for additional 7-15 times.

The improvement of the ulcers is clearly demonstrated in FIGS. 1B-3B.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A composition comprising as an active ingredient an oil extract or oily suspension of a combination of ground roots of Harpagophytum procumbens (Devil's claw) and Angelica archangelica (Angelica).

2. The composition of claim 1, wherein the active ingredient further comprises an oil extract or oily suspension of ground roots of Hydrastis canadensis (Golden Seal).

3. The composition of claim 1, further comprising an oil extract or oily suspension of at least one of ground root of Astragalus membranaceous (Astragalus); ground leaves of Symphytum officinale (Comfrey) ground leaves of Urtica dioica (Stinging Nettle) or any combination thereof.

4. The Composition of claim 3, further comprising an oil extract or oily suspension of ground leaves of Agrimonia Eupatoria (Agrimony) and ground blue-green algae.

5. The composition of claim 4 comprising as an active ingredient an oil extract or oily suspension of a combination of ground plant material comprising roots of Harpagophytum procumbens (Devil's claw), Angelica Archangelica (Angelica) and Astragalus membranaceous (Astragalus); leaves of Symphytum officinale (Comfrey), Urtica dioica (Stinging Nettle) and Agrimonia Eupatoria (Agrimony); and blue-green algae.

6. The composition of claim 5, wherein the active ingredient further comprises an oil extract or oily suspension of ground roots of Hydrastis Canadensis (Golden Seal).

7. The composition of claim 5, consisting of an oil extract or oily suspension of a combination of ground plant material consisting of roots of Harpagophytum procumbens (Devil's claw), Angelica Archangelica (Angelica) and Astragalus membranaceous (Astragalus); leaves of Symphytum officinale (Comfrey), Urtica dioica (Stinging Nettle) and Agrimonia Eupatoria (Agrimony); and ground blue-green algae.

8. A composition consisting of an oil extract or oily suspension of a combination of ground plant material consisting of roots of Harpagophytum procumbens (Devil's claw), Angelica Archangelica (Angelica), Hydrastis Canadensis (Golden Seal), and Astragalus membranaceous (Astragalus); leaves of Symphytum officinale (Comfrey), Urtica dioica (Stinging Nettle) and Agrimonia Eupatoria (Agrimony); and ground blue-green algae.

9. The composition of claim 1, wherein the oil is selected from the group consisting of a mineral oil, a vegetable oil and combinations thereof.

10. The composition of claim 9, wherein the oil is a vegetable oil selected from the group consisting of wheat germ oil, almond oil, canola oil, coconut oil, corn oil, cottonseed oil, grape seed oil, olive oil peanut oil, saffron oil, sesame oil, soybean oil, and combinations thereof.

11. The composition of claim 10, wherein the oil is wheat germ oil.

12. The composition of claim 1, wherein the plant material is in a dry form.

13. The composition of claim 12, wherein the dry material is ground to a fine powder.

14. The composition of claim 1, further comprising at least one additional active ingredient selected from the group consisting of an anti-inflammatory agent, analgesic agent, anesthetic agent, antihistamine, disinfectant, antibiotic agent and a drug effective in wound healing.

15. A pharmaceutical or dermatological composition comprising the composition of claim 1, further comprising a pharmaceutically or dermatologically acceptable diluent, excipient or carrier.

16. The pharmaceutical or dermatological composition of claim 15, further comprising an additive selected from the group consisting of fats, emulsifiers, co-emulsifiers, hydrophilic or lipophilic gelling agents, preservatives, solvents, fragrances, fillers, hydrophilic and lipophilic filters, neutralizers, penetration-enhancing agents and polymers.

17. The pharmaceutical or dermatological composition of claim 15, formulated for topical administration onto a wound.

18. The pharmaceutical or dermatological composition of claim 15, formulated for topical administration by injection into a wound.

19. A method for treating a burn or chronic wound comprising topically administering to a subject in need thereof an effective amount of a composition according to claim 15.

20. The method of claim 19, further comprising covering the treated wound or burn area with a wound dressing.

21-29. (canceled)
